(19) United States
(12) Patent Application Publication
(10) Pub. No.: US 2021/0244843 A1
(43) Pub. Date: Aug. 12, 2021

(54) Self-Extinguishing Safety Scent Stick

(71) Applicant: William Michael Long, Jacksonville, FL (US)

(72) Inventor: William Michael Long, Jacksonville, FL (US)

(21) Appl. No.: 16/785,220

(22) Filed: Feb. 7, 2020

(51) Int. Cl. A61L 9/03

(52) U.S. Cl. CPC A61L 9/03

(57) ABSTRACT

The present invention comprises an improvement that embodies an innovation in fire safety. As compared to the typical linear “incense stick”, it is self-extinguishing after releasing a controlled amount of scent, without employing any additional apparatus or fire retardant. Consumers may also save money by not neglecting to extinguish the stick, releasing more scent than desired. Burn times are determined by the length and diameter of the combustible sections, being determined by the manufacturer.

### FIELD OF THE INVENTION

The present invention relates to the scent dispersal industry, or “air freshener” or “odor masking” industry. In particular, to what is commonly referred to as a linear “incense” stick or a “scent stick”, comprised of combustible material, treated with aromatic compounds, applied to a (typically) thin bamboo “stick”. The “stick” is ignited, and burns, releasing fragrant smoke.

### BACKGROUND ART

“Freshening” the air inside a domicile or other enclosed space, or the “masking” of undesirable odors is a problem as old as humankind

Many forms of “air fresheners” are marketed currently. These include, but are not limited to, aerosol dispersal of scented oils by propellant or pump spray, scented candles, potpourri, low-wattage electrically warmed aromatics, and linear “incense” or “scent sticks” which are ignited and burned.

All forms of “air fresheners” have inherent drawbacks, including, but not limited to, inhalation hazards and environmental problems of disposal, but three of the aforementioned methods (low-wattage electrically warmed aromatics, scented candles and linear “incense” or “scent sticks”) also carry the risk of fire.

There is also a possible excess expenditure of funds by wasting quantities of scent in excess of what is needed to achieve the desired odor masking effect, due to neglecting to extinguish a burning linear “incense” or “scent stick”, as it will burn until the combustible material is consumed.

Low-wattage electrically warmed scents are the least dangerous in terms of fire hazard and monetary waste. While not particularly difficult to extinguish, scented candles and “incense” or “scent sticks” have been left unattended, resulting in conflagrations by contact with other combustibles.

### BRIEF SUMMARY OF THE INVENTION

The present invention comprises an improvement to the already existing linear “incense” or “scent stick” in that it embodies the incorporation of intermittent “fire breaks”, where the combustible material has either been removed or never was applied to those sections.

### BRIEF DESCRIPTION OF THE DRAWINGS

FIG. 1 illustrates the typical linear “scent stick”, with combustible material covering from one end tip(which is ignited) to a point where the support stick is meant to be placed into a holder.

FIG. 2-F illustrates a linear “scent stick” into which has been introduced a series of intermittent “fire breaks”.

FIG. 3.- Depicts a side profile view of a typical linear “scent stick” with the fire breaks, formed by mechanical removal of the combustible scent material.

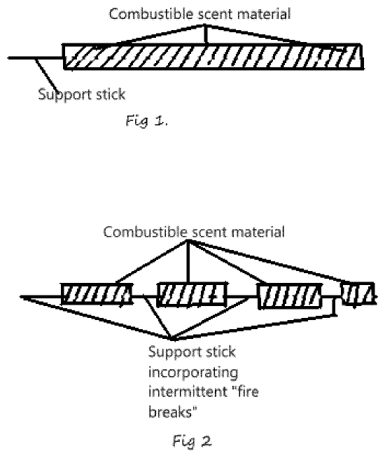

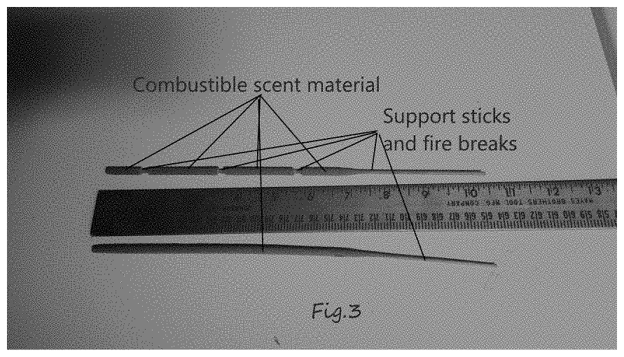

### DETAILED DESCRIPTION OF THE INVENTION

The present invention embodies any linear “scent stick” of any length and diameter, into which a series of “fire breaks” has been intoduced at pre-determined intervals, providing controlled lenghts of combustion and scent release in relation to time. The “fire breaks” may be incorporated either while the stick is being formed, or the combustible material may be removed mechanically after formation of the stick. The introduction of the aforementioned “fire breaks” renders the linear “incense stick” to become “self-extinguishing” after a controlled release of scent. Typically, no matter the length or diameter of the linear “scent stick” a fire break of two millimeters (2mm) is all that is required for the self-extinguishing characteristic to be effective.

What is claimed is:

1. A reduced risk of fire resulting from an unattended linear “incense stick”;

2. A consumer savings in terms of controlled release of scent, and less waste due to neglecting to extinguish a linear “incense stick”;

3. No additional fire retardant chemicals or apparatus need be added to achieve the self-extiguishing characteristic.
